(19) United States
(12) Patent Application Publication
(10) Pub. No.: US 2005/0064011 A1
(43) Pub. Date: Mar. 24, 2005

(54) Implantable or insertable medical devices containing phenolic compound for inhibition of restenosis

(76) Inventors: Young-Ho Song, Framingham, MA (US); Andrew J. Carter, Portage, MI (US)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/955,368

(22) Filed: Sep. 30, 2004

Related U.S. Application Data
(63) Continuation-in-part of application No. 10/638,920, filed on Aug. 11, 2003.

(51) Int. Cl. A61F 2/00

(52) U.S. Cl. 424/423

(57) ABSTRACT

A vascular medical device is provided, which contains at least one phenolic compound. The medical device also contains at least one polymeric region, which regulate the release of the phenolic compound from the device. The polymeric region, in turn, contains at least one polymer species. In some embodiments, for example, the polymeric region contains a vinyl aromatic polymer species (e.g., a styrene homopolymer or copolymer). In other embodiments, for example, the polymeric region contains an alkene polymer species (e.g., an isobutylene homopolymer or copolymer). In still other embodiments, for example, the polymeric region contains a biostable polymer having at least one Tg below 25° C. (e.g., a homopolymer or copolymer containing one or more polyalkene polymer blocks).

### STATEMENT OF RELATED APPLICATION

This application is a continuation-in-part of U.S. application Ser. No. 10/638,920, entitled “Medical Devices Containing Antioxidant and Therapeutic Agent,” filed Aug. 11, 2003, which is incorporated by reference in its entirety herein.

### FIELD OF THE INVENTION

The present invention relates to radiation-resistant implantable or insertable medical devices.

### BACKGROUND OF THE INVENTION

It is known to use polymers in conjunction with implantable or insertable medical devices, for example, as coatings for medical devices. However, such polymers frequently elicit a vigorous immune or foreign body response. This is particularly true of intravascular or intervascular medical devices, which commonly suffer from the consequences of inflammation and neointimal thickening after placement within the vasculature.

Polymers are known, for example, the polystyrene-polyisobutylene block copolymers described in U.S. Pat. No. 6,545,097 to Pinchuk et al., which have a tendency to provoke minimal adverse reactions within the body. However, some degree of inflammation and neointimal thickening is nonetheless observed, even for these polymers.

Accordingly, there is an ongoing need in the art for strategies by which adverse reactions associated with various polymeric materials are reduced.

Percutaneous transluminal coronary angioplasty (“PTCA” or “angioplasty”) procedures have been performed for many years as an adjunct to correcting vascular disease in patients. Angioplasty procedures typically involve the insertion, through the vascular system, of a catheter having a balloon that is placed across a lesion or blockage in a coronary artery. The balloon is then inflated to compress the lesion or blockage against the arterial walls, thereby opening the artery for increased blood flow. In some cases, however, the goal of the angioplasty procedure is defeated at least in part by a complete or partial reclosure of the artery at or near the compressed lesion or blockage due to restenosis.

Accordingly, there is an ongoing need in the art for strategies by which adverse reactions associated with the implantation or insertion of medical devices into the vasculature, including restenosis, are reduced.

### SUMMARY OF THE INVENTION

The above and other needs are met by the present invention in which a vascular medical device is provided, which contains at least one phenolic compound. The medical device also contains at least one polymeric region, which regulates the release of the phenolic compound from the device. The polymeric region, in turn, contains at least one polymer species.

In some embodiments, for example, the polymeric region contains a vinyl aromatic polymer species (e.g., a styrene homopolymer or copolymer). In other embodiments, for example, the polymeric region contains an alkene polymer species (e.g., an isobutylene homopolymer or copolymer). In still other embodiments, for example, the polymeric region contains a biostable polymer having at least one Tg below 25° C. (e.g., a homopolymer or copolymer containing one or more polyalkene polymer blocks).

The present invention is advantageous in that adverse reactions, which are frequently reported in conjunction with the implantation or insertion of medical devices into the vasculature (e.g., restenosis), are reduced.

Another advantage of the present invention is that the biocompatibility of various polymers, even polymers known for their outstanding biocompatibility, can be improved.

These and other embodiments and advantages of the present invention will become immediately apparent to those of ordinary skill in the art upon review of the Detailed Description and claims to follow.

### DETAILED DESCRIPTION OF THE INVENTION

According to an aspect of the present invention, vascular medical devices are provided, which contain one or more phenolic compounds. The phenolic compounds are typically released from the devices in amounts effective to reduce the amount of neointimal hyperplasia that is associated with the insertion or implantation of the medical devices into the vasculature. The medical devices also contain one or more polymeric regions, which contain one or more polymer species and which typically regulate the release of the phenolic compound from the devices.

Vascular medical devices benefiting from the present invention include intravascular and intervascular devices such as catheters (e.g., expandable catheters such as balloon catheters), guide wires, balloons, filters (e.g., vena cava filters), stents (including coronary vascular stents and cerebral stents), cerebral aneurysm filler coils (including Guglilmi detachable coils and metal coils), vascular grafts, stent grafts, myocardial plugs, patches, pacemakers and pacemaker leads, heart valves, sutures, suture anchors, anastomosis clips and rings, tissue staples and ligating clips at surgical sites, tissue engineering scaffolds, or any other medical device for implantation or insertion into the heart, coronary vascular system and peripheral vascular system (referred to overall as “the vasculature”).

One particularly preferred medical device for use in conjunction with the present invention is a coated vascular stent, which provides treatment for restenosis. As used herein, “treatment” refers to the prevention of a disease or condition, the reduction or elimination of symptoms associated with a disease or condition, or the substantial or complete elimination a disease or condition. Preferred subjects are mammalian subjects and more preferably human subjects.

“Phenolic compounds,” as defined herein, are compounds which contain a six sided aromatic ring (which ring can be part of a multi-cyclic ring system) having at least one pendent alcohol group. Phenolic compounds for the practice of the present invention can be selected, for example, from one or more of the following: hindered phenols such as butylated hydroxyanisole (BHA) and butylated hydroxytoluene (BHT), and polyphenolic compounds such as probucol; hydroquinones such as methyl hydroquinone, tertiary-butyl hydroquinone (TBHQ) and 1-O-hexyl-2,3,5-trimethyl hydroquinone (HTHQ); nordihydroguaiaretic acid (NDGA); alkoxyphenols such as 4-tert-butoxyphenol, 4-ethoxyphenol, 3-methoxyphenol and 2-tert-butyl-4-methoxyphenol; 2,2-methylene-bis-(4-methyl-6-tert-butylphenol); tocopherols such as alpha-tocopherol (vitamin E), beta-tocopherol, gamma-tocopherol and delta-tocopherol; phenolic acids and their esters including para-coumaric acid, caffeic acid, chlorogenic acid, ferulic acid, protocatechuic acid, cinnamic acid, gallic acid, alkyl gallates (e.g., propyl, octyl, dodecyl), and para-hydroxybenzoic acid. Other phenolic compounds include flavonoids, such as catechins, leucoanthocyanidins, flavanones, flavanins, flavones, anthocyanins, flavonols, flavones, isoflavones, proanthocyanidins, flavonoid, pyrocatechol derivatives, and so forth. Specific examples are catechin, quercetin and rutin.

The phenolic compounds that are used in conjunction with the present invention are beneficially phenolic compounds approved by the United States Food and Drug Administration (USFDA) for use in food and/or drugs.

Phenolic compounds can be disposed upon or within the medical devices of the present invention using a variety of schemes. For example, in some embodiments, a phenolic compound is disposed within or beneath the polymeric region that is associated with the medical device. In these embodiments, the polymeric region can constitute the entirety of the medical device, or only a portion thereof. For example, in many embodiments, the polymeric region is in the form of a layer, which may be disposed over the entirety of an underlying medical device substrate or over only a portion thereof. The underlying substrate can comprise, for example, metal, ceramic and polymeric materials such as those discussed elsewhere herein. As used herein a “layer” of a given material is a region of that material whose thickness is small compared to both its length and width. As used herein a layer need not be planar, for example, taking on the contours of an underlying substrate. Layers can be discontinuous (e.g., patterned). Terms such as “film,” “layer” and “coating” may be used interchangeably herein.

In some embodiments, the polymeric region is a polymeric release layer that acts to control the release of the phenolic compound upon administration to a patient. By “release layer” is meant a layer that regulates the rate of release of the phenolic compound. Release layers are commonly either carrier layers or barrier layers. A “carrier layer” is a layer which contains the at least one phenolic compound and from which the phenolic compound is released. A “barrier layer” is a layer that is disposed between a source of the phenolic compound and a site of intended release, which controls the rate at which the phenolic compound is released.

As noted above, substrates, where utilized, include ceramic substrates, metallic substrates, polymeric substrates, and combinations of the same. Ceramic materials can be selected, for example, from materials comprising one or more of the following: metal oxides, including aluminum oxides and transition metal oxides (e.g., oxides of titanium, zirconium, hafnium, tantalum, molybdenum, tungsten, rhenium, and iridium); silicon-based ceramics, such as those containing silicon nitrides, silicon carbides and silicon oxides (sometimes referred to as glass ceramics); calcium phosphate ceramics (e.g., hydroxyapatite); and carbon-based ceramic-like materials such as carbon nitrides. Metallic materials (which may or may not have a natural or man-made native oxide surface) can be selected, for example, from materials comprising one or more of the following: noble metals such as silver, gold, platinum, palladium, iridium, osmium, rhodium, titanium, tungsten, and ruthenium, metal alloys such as cobalt-chromium alloys, nickel-titanium alloys (e.g., nitinol), cobalt-chromium-iron alloys (e.g., elgiloy alloys), nickel-chromium alloys (e.g., inconel alloys), and iron-chromium alloys (e.g., stainless steels, which contain at least 50% iron and at least 11.5% chromium). Polymeric materials for use as substrates can be selected, for example, from materials comprising one or more of the polymer listed below in conjunction with the polymeric regions of the present invention.

A variety of polymers can be used in conjunction with the polymeric regions of the present invention, including homopolymers and copolymers (e.g., alternating, random, statistical, gradient and block copolymers); cyclic, linear and branched polymers (e.g., polymers having star, comb and dendritic architectures); natural and synthetic polymers; thermoplastic and thermosetting polymers; and so forth. Specific examples of polymers for the practice of the invention may be selected, for example, from the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, vinyl aromatic polymers and copolymers such as polystyrenes, styrene-maleic anhydride copolymers, vinyl aromatic-hydrocarbon copolymers including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); polyesters including polyethylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as blends and copolymers of the above.

In some beneficial embodiments of the invention, the polymeric regions include at least one polymer that contains an alkene monomer, a vinyl aromatic monomer, or both. Specific examples are copolymers containing one or more alkene monomers as well as one or more vinyl aromatic monomers. These copolymers include, for example, alternating, random, statistical, gradient and block copolymers, and they can have a variety of architectures, for example, cyclic, linear and branched (e.g., star, comb or dendritic) architectures. Specific examples include polystyrene-polyisobutylene block copolymers, for example, polystyrene-polyisobutylene-polystyrene, which is a linear triblock copolymer.

In some beneficial embodiments of the invention, the polymeric regions include homopolymers and copolymers that contain at least one low T_{g} polymer block. As used herein, a polymer “block” is a grouping of 10 or more constitutional units (i.e., monomers), commonly 20 or more, 50 or more, 100 or more, 200 or more, 500 or more, or even 1000 or more units. A “chain” is a linear (unbranched) grouping of 10 or more constitutional units (i.e., a linear block).

A “low T_{g} polymer block” is a polymer block that displays one or more glass transition temperatures (T_{g}), as measured by any of a number of techniques including differential scanning calorimetry (DSC), dynamic mechanical analysis (DMA), or dielectric analysis (DEA), that is below ambient temperature, more typically below 25° C., below 0° C., below −25° C., or even below −50° C. “Ambient temperature” is typically 25° C.-45° C., more typically body temperature (e.g., 35° C.-40° C.). As a result of their low glass transition temperatures, low T_{g} polymer blocks are typically elastomeric at ambient temperature. Homopolymers of some low T_{g} polymer blocks, such as linear or branched silicone (e.g. polydimethylsiloxane), are viscous liquids or millable gums at room temperature and become elastomeric upon covalent cross-linking.

Conversely, an elevated or “high T_{g} polymer block” is a polymer block that displays one or more glass transition temperatures, as measured by any of a number of techniques including differential scanning calorimetry, dynamic mechanical analysis, or thermomechanical analysis, which is above ambient temperature, more typically above 50° C., above 60° C., above 70° C., above 80° C., above 90° C. or even above 100° C.

Hence, copolymers having one or more low T_{g} blocks and one or more high T_{g} polymer blocks will have one or more glass transition temperatures below ambient temperature and one or more glass transition temperatures above ambient temperature. This typically results in the formation of rubbery and hard phases within the coating layer at ambient temperatures.

Low and high T_{g} polymer blocks may be provided in a variety of configurations, including cyclic, linear and branched configurations. Branched configurations include star-shaped configurations (e.g., configurations in which three or more chains emanate from a single branch point), comb configurations (e.g., configurations having a main chain and a plurality of branching side chains) and dendritic configurations (e.g., arborescent and hyperbranched polymers). The low and high T_{g} polymer blocks may contain, for example, a repeating series of units of a single type, a series of units of two or more types in a repeating (e.g., alternating), random, statistical or gradient distribution, and so forth.

Specific examples of low T_{g} polymer blocks from which the low T_{g} polymer blocks of the present invention can be selected include homopolymers and copolymer blocks containing one or more of the following constitutional units: acrylic monomers, methacrylic monomers, vinyl ether monomers, cyclic ether monomers, ester monomers, unsaturated hydrocarbon monomers, including alkene monomers, halogenated alkene monomers, halogenated unsaturated hydrocarbon monomers, and siloxane monomers. Numerous specific examples are listed below.

Note that a polymer described herein as “containing a monomer” or “including a monomer” or “comprising a monomer,” is one that is either formed using such a monomer, or has the appearance of being formed using such a monomer. For example, polymers that comprise styrene monomer (e.g., polystyrene homopolymers and copolymers) are typically formed using styrene as a monomer. In contrast, polymers that comprise vinyl alcohol (e.g., poly(vinyl alcohol) homopolymers and copolymers) are not actually formed using vinyl alcohol, which is an unstable liquid, but rather have the appearance of being formed from vinyl alcohol.

With that understanding, specific low T_{g} acrylic monomers (i.e., acrylic monomers that may be used to form low T_{g} polymer blocks) include the following (the T_{g} values are published values for homopolymers of the listed monomer): (a) alkyl acrylates such as methyl acrylate (T_{g} 10° C.), ethyl acrylate (T_{g} −24° C.), propyl acrylate, isopropyl acrylate (T_{g} −11° C., isotactic), butyl acrylate (T_{g} −54° C.), sec-butyl acrylate (T_{g} −26° C.), isobutyl acrylate (T_{g} −24° C.), cyclohexyl acrylate (T_{g} 19° C.), 2-ethylhexyl acrylate (T_{g} −50° C.), dodecyl acrylate (T_{g} −3° C.) and hexadecyl acrylate (T_{g} 35° C.), (b) arylalkyl acrylates such as benzyl acrylate (T_{g} 6° C.), (c) alkoxyalkyl acrylates such as 2-ethoxyethyl acrylate (T_{g} −50° C.) and 2-methoxyethyl acrylate (T_{g} −50° C.), (d) halo-alkyl acrylates such as 2,2,2-trifluoroethyl acrylate (T_{g} −10° C.) and (e) cyano-alkyl acrylates such as 2-cyanoethyl acrylate (T_{g} 4° C.).

Specific low T_{g} methacrylic monomers include the following: (a) alkyl methacrylates such as butyl methacrylate (T_{g} 20° C.), hexyl methacrylate (T_{g} −5° C.), 2-ethylhexyl methacrylate (T_{g} −10° C.), octyl methacrylate (T_{g} −20° C.), dodecyl methacrylate (T_{g} −65° C.), hexadecyl methacrylate (T_{g} 15° C.) and octadecyl methacrylate (T_{g} −100° C.) and (b) aminoalkyl methacrylates such as diethylaminoethyl methacrylate (T_{g} 20° C.) and 2-tert-butyl-aminoethyl methacrylate (T_{g} 33° C.).

Specific low T_{g} vinyl ether monomers include the following: (a) alkyl vinyl ethers such as methyl vinyl ether (T_{g} −31° C.), ethyl vinyl ether (T_{g} −43° C.), propyl vinyl ether (T_{g} −49° C.), butyl vinyl ether (T_{g} −55° C.), isobutyl vinyl ether (T_{g} −19° C.), 2-ethylhexyl vinyl ether (T_{g} −66° C.) and dodecyl vinyl ether (T_{g} −62° C.).

Specific low T_{g} acyclic ether monomers include the following: tetrahydrofuran (T_{g} −84° C.), trimethylene oxide (T_{g} −78° C.), ethylene oxide (T_{g} −66° C.), propylene oxide (T_{g} −75° C.), methyl glycidyl ether (T_{g} −62° C.), butyl glycidyl ether (T_{g} −79° C.), allyl glycidyl ether (T_{g} −78° C.), epibromohydrin (T_{g} −14° C.), epichlorohydrin (T_{g} −22° C.), 1,2-epoxybutane (T_{g} −70° C.), 1,2-epoxyoctane (T_{g} −67° C.) and 1,2-epoxydecane (T_{g} −70° C.).

Specific low T_{g} ester monomers (other than acrylates and methacrylates) include the following: ethylene malonate (T_{g} −29° C.), vinyl acetate (T_{g} 30° C.), and vinyl propionate (T_{g} 10° C.).

Specific low T_{g} alkene monomers include the following: ethylene, propylene (T_{g} −8 to −13° C.), isobutylene (T_{g} −73° C.), 1-butene (T_{g} −24° C.), trans-butadiene (T_{g} −58° C.), 4-methyl pentene (T_{g} 29° C.), 1-octene (T_{g} −63° C.) and other α-olefins, cis-isoprene (T_{g} −63° C.), and trans-isoprene (T_{g} −66° C.).

Specific low T_{g} halogenated alkene monomers include the following: vinylidene chloride (T_{g} −18° C.), vinylidene fluoride (T_{g} −40° C.), cis-chlorobutadiene (T_{g} −20° C.), and trans-chlorobutadiene (T_{g} −40° C.).

Specific low T_{g} siloxane monomers include the following: dimethylsiloxane (T_{g} −127° C.), diethylsiloxane, methylethylsiloxane, methylphenylsiloxane (T_{g} −86° C.), and diphenylsiloxane.

Specific examples of high T_{g} polymer blocks include homopolymer and copolymer blocks containing (i.e., formed from or having the appearance of being formed from) the following monomers: various vinyl aromatic monomers, other vinyl monomers, other aromatic monomers, methacrylic monomers, and acrylic monomers. Numerous specific examples are listed below. The T_{g} values are published values for homopolymers of the listed monomer.

Vinyl aromatic monomers are monomers having aromatic and vinyl moieties, including unsubstituted monomers, vinyl-substituted monomers and ring-substituted monomers. Several specific high T_{g} vinyl aromatic monomers follow: (a) unsubstituted vinyl aromatics, such as atactic styrene (T_{g} 100° C.), isotactic styrene (T_{g} 100° C.) and 2-vinyl naphthalene (T_{g} 151° C.), (b) vinyl substituted aromatics such as methyl styrene, (c) ring-substituted vinyl aromatics including (i) ring-alkylated vinyl aromatics such as 3-methylstyrene (T_{g} 97° C.), 4-methylstyrene (T_{g} 97° C.), 2,4-dimethylstyrene (T_{g} 112° C.), 2,5-dimethylstyrene (T_{g} 143° C.), 3,5-dimethylstyrene (T_{g} 104° C.), 2,4,6-trimethylstyrene (T_{g} 162° C.), and 4-tert-butylstyrene (T_{g} 127° C.), (ii) ring-alkoxylated vinyl aromatics, such as 4-methoxystyrene (T_{g} 113° C.) and 4-ethoxystyrene (T_{g} 86° C.), (iii) ring-halogenated vinyl aromatics such as 2-chlorostyrene (T_{g} 119° C.), 3-chlorostyrene (T_{g} 90° C.), 4-chlorostyrene (T_{g} 110° C.), 2,6-dichlorostyrene (T_{g} 167° C.), 4-bromostyrene (T_{g} 118° C.) and 4-fluorostyrene (T_{g} 95° C.) and (iv) ester-substituted vinyl aromatics such as 4-acetoxystyrene (T_{g} 116° C.).

Other specific high T_{g} vinyl monomers include: (a) vinyl alcohol (T_{g} 85° C.); (b) vinyl esters such as vinyl benzoate (T_{g} 71° C.), vinyl 4-tert-butyl benzoate (T_{g} 101° C.), vinyl cyclohexanoate (T_{g} 76° C.), vinyl pivalate (T_{g} 86° C.), vinyl trifluoroacetate (T_{g} 46° C.), vinyl butyral (T_{g} 49° C.), (c) vinyl amines such as 2-vinyl pyridine (T_{g} 104° C.), 4-vinyl pyridine (T_{g} 142° C.), and vinyl carbazole (T_{g} 227° C.), (d) vinyl halides such as vinyl chloride (T_{g} 81° C.) and vinyl fluoride (T_{g} 40° C.); (e) alkyl vinyl ethers such as tert-butyl vinyl ether (T_{g} 88° C.) and cyclohexyl vinyl ether (T_{g} 81° C.), and (f) other vinyl compounds such as 1-vinyl-2-pyrrolidone (T_{g} 54° C.) and vinyl ferrocene (T_{g} 189° C.).

Specific high T_{g} aromatic monomers, other than vinyl aromatics, include: acenaphthalene (T_{g} 214° C.) and indene (T_{g} 85° C.).

Specific high T_{g} methacrylic monomers include (a) methacrylic acid (T_{g} 228° C.), (b) methacrylic acid salts such as sodium methacrylate (T_{g} 310° C.), (c) methacrylic acid anhydride (T_{g} 159° C.), (d) methacrylic acid esters (methacrylates) including (i) alkyl methacrylates such as atactic methyl methacrylate (T_{g} 105-120° C.), syndiotactic methyl methacrylate (T_{g} 115° C.), ethyl methacrylate (T_{g} 65° C.), isopropyl methacrylate (T_{g} 81° C.), isobutyl methacrylate (T_{g} 53° C.), t-butyl methacrylate (T_{g} 118° C.) and cyclohexyl methacrylate (T_{g} 92° C.), (ii) aromatic methacrylates such as phenyl methacrylate (T_{g} 110° C.) and including aromatic alkyl methacrylates such as benzyl methacrylate (T_{g} 54° C.), (iii) hydroxyalkyl methacrylates such as 2-hydroxyethyl methacrylate (T_{g} 57° C.) and 2-hydroxypropyl methacrylate (T_{g} 76° C.), (iv) additional methacrylates including isobornyl methacrylate (T_{g} 110° C.) and trimethylsilyl methacrylate (T_{g} 68° C.), and (e) other methacrylic-acid derivatives including methacrylonitrile (T_{g} 120° C.).

Specific high T_{g} acrylic monomers include (a) acrylic acid (T_{g} 105° C.), its anhydride and salt forms, such as potassium acrylate (T_{g} 194° C.) and sodium acrylate (T_{g} 230° C.); (b) certain acrylic acid esters such as tert-butyl acrylate (T_{g} 43-107° C.) (Tₘ 193° C.), hexyl acrylate (T_{g} 57° C.) and isobornyl acrylate (T_{g} 94° C.); (c) acrylic acid amides such as acrylamide (T_{g} 165° C.), N-isopropylacrylamide (T_{g} 85-130° C.) and N,N dimethylacrylamide (T_{g} 89° C.); and (d) other acrylic-acid derivatives including acrylonitrile (T_{g} 125° C.).

Numerous copolymers containing both low and high T_{g} polymer blocks are known, including copolymers that contain one or more vinyl aromatic blocks as well as one or more alkene blocks, such as polystyrene-poly(ethylene/butylene)-polystyrene (SEBS) copolymers, available as Kraton® G series polymers, and polyisobutylene-polystyrene-polyisobutylene (SIBS) copolymers, described, for example, in U.S. Pat. No. 6,545,097 to Pinchuk et al.

Numerous techniques are available for forming polymeric regions for the practice of the present invention. For example, where one or more polymers that are selected to form the polymeric regions have thermoplastic characteristics, a variety of standard thermoplastic processing techniques can be used, including compression molding, injection molding, blow molding, spinning, vacuum forming and calendaring, as well as extrusion into sheets, fibers, rods, tubes and other cross-sectional profiles of various lengths. Using these and other techniques, entire devices or portions thereof can be made. For example, an entire stent can be extruded using the above techniques. As another example, a coating can be provided by extruding a coating layer onto a pre-existing stent. As yet another example, a coating can be co-extruded, along with an underlying stent body.

Where the phenolic compound and any optional supplemental agents (e.g., therapeutic agents such as those listed below) are stable at processing temperatures, then they can be combined with the one or more polymers prior to thermoplastic processing. If not, then they can nonetheless be introduced subsequent to thermoplastic processing, for example, using techniques such as those discussed below.

Polymeric regions can also be formed using solvent-based techniques in which one or more polymers comprising the polymeric region are first dissolved or dispersed in a solvent and the resulting mixture subsequently used to form the polymeric region.

Where solvent-based techniques are used, the solvent system that is selected will contain one or more solvent species. The solvent system preferably is a good solvent for the one or more polymers forming the polymeric region and, where included, for the one or more phenolic compounds and any optional supplemental agents. The particular solvent species that make up the solvent system may also be selected based on other characteristics including drying rate and surface tension.

Preferred solvent-based techniques include, but are not limited to, solvent casting techniques, spin coating techniques, web coating techniques, solvent spraying techniques, dipping techniques, techniques involving coating via mechanical suspension including air suspension, ink jet techniques, electrostatic techniques, and combinations of these processes.

In many embodiments, a mixture containing solvent, one or more polymers (and, if desired, one or more phenolic compounds and any optional supplemental agents) is applied to a substrate to form a polymeric region. For example, the substrate can be all or a portion of a medical device, such as a stent, to which a polymeric layer is applied. On the other hand, the substrate can also be, for example, a template, such as a mold, from which the polymeric region is removed after solvent elimination. Such template-based techniques are particularly appropriate for forming simple objects such as sheets, tubes, cylinders and so forth, which can be easily removed from a template substrate.

In other techniques, for example, fiber forming techniques, the polymeric region is formed without the aid of a substrate or template.

Where appropriate, techniques such as those listed above can be repeated or combined to build up a polymeric region to a desired thickness. The thickness of the polymeric region can be varied in other ways as well. For example, in one preferred process, solvent spraying, coating thickness can be increased by modification of coating process parameters, including increasing spray flow rate, slowing the movement between the substrate to be coated and the spray nozzle, providing repeated passes and so forth.

As indicated above, in some embodiments, the one or more phenolic compounds and/or any optional supplemental agents are combined with the one or more polymers during solvent based processing and hence co-established with the polymeric region. In other embodiments, on the other hand, the one or more phenolic compounds and/or any optional supplemental agents are dissolved within a solvent, and the resulting solution contacted, for example, using one or more of the application techniques described above (e.g., dipping, spraying, etc.) with a previously formed polymeric region.

In some embodiments, a barrier layer is formed over a region that contains one or more phenolic compounds (and any optional therapeutic agents) using, for example, solvent-based techniques such as those discussed above. For instance, one or more polymers (and any additional agents, where desired) can be first dissolved or dispersed in a solvent, and the resulting mixture subsequently used to form the barrier layer. The barrier layer serves, for example, as a boundary layer to retard diffusion of the underlying one or more phenolic compounds (and any optional therapeutic agents) acting to prevent, for example, a burst phenomenon whereby much of the one or more phenolic compounds (and any optional therapeutic agents) are released immediately upon exposure of the device or a portion of the device to the implant or insertion site. In some embodiments, the region beneath the barrier region that contains the one or more phenolic compounds (and any optional therapeutic agents) will comprise one or more polymers such as those described elsewhere herein. In these embodiments, the polymeric composition of the barrier region may, or may not, be the same as the polymeric composition of the underlying region. In other embodiments, the therapeutic-agent-containing region beneath the barrier layer is established without an associated polymer. For example, the one or more phenolic compounds (and any optional therapeutic agents) can simply be dissolved or dispersed in a solvent or liquid, and the resulting solution/dispersion can be contacted with a substrate (using one or more of the above-described application techniques, for instance).

Where the polymeric region is formed using a solvent-based technique, it is preferably dried after application to remove the solvents. Where a medical device substrate is coated, the polymeric layer that is formed typically further conforms to the substrate during the drying process.

Supplemental therapeutic agents may be optionally used singly or in combination in the medical devices of the present invention. “Drugs,” “therapeutic agents,” “pharmaceutically active agents,” “pharmaceutically active materials,” and other related terms may be used interchangeably herein. These terms include genetic therapeutic agents, non-genetic therapeutic agents and cells.

Exemplary non-genetic therapeutic agents for use in conjunction with the present invention include: (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines; and (r) hormones.

Preferred non-genetic therapeutic agents include paclitaxel, sirolimus, everolimus, tacrolimus, dexamethasone, estradiol, ABT-578 (Abbott Laboratories), trapidil, liprostin, Actinomcin D, Resten-NG, Ap-17, abciximab, clopidogrel and Ridogrel.

Exemplary genetic therapeutic agents for use in conjunction with the present invention include anti-sense DNA and RNA as well as DNA coding for the various proteins (as well as the proteins themselves): (a) anti-sense RNA, (b) tRNA or rRNA to replace defective or deficient endogenous molecules, (c) angiogenic and other factors including growth factors such as acidic and basic fibroblast growth factors, vascular endothelial growth factor, endotherial mitogenic growth factors, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PEI and polyethylene oxide-PEI), neutral polymers PVP, SP1017 (SUPRATEK), lipids such as cationic lipids, liposomes, lipoplexes, nanoparticles, or microparticles, with and without targeting sequences such as the protein transduction domain (PTD).

Cells for use in conjunction with the present invention include cells of human origin (autologous or allogeneic), including whole bone marrow, bone marrow derived mono-nuclear cells, progenitor cells (e.g., endothelial progenitor cells), stem cells (e.g., mesenchymal, hematopoietic, neuronal), pluripotent stem cells, fibroblasts, myoblasts, satellite cells, pericytes, cardiomyocytes, skeletal myocytes or macrophage, or from an animal, bacterial or fungal source (xenogeneic), which can be genetically engineered, if desired, to deliver proteins of interest.

Numerous therapeutic agents, not necessarily exclusive of those listed above, have been identified as candidates for vascular treatment regimens, for example, as agents targeting restenosis. Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) ACE inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (I) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD mimics, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) MMP pathway inhibitors such as marimastat, ilomastat and metastat, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, paclitaxel and epothilone), caspase activators, proteasome inhibitors, angiogenesis inhibitors (e.g., endostatin, angiostatin and squalamine), rapamycin, cerivastatin, flavopiridol and suramin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Numerous additional therapeutic agents useful for the practice of the present invention are also disclosed in U.S. Pat. No. 5,733,925 assigned to NeoRx Corporation, the entire disclosure of which is incorporated by reference.

A wide range of therapeutic agent loadings can be used in conjunction with the medical devices of the present invention, with the therapeutically effective amount being readily determined by those of ordinary skill in the art and ultimately depending, for example, upon the condition to be treated, the age, sex and condition of the patient, the nature of the therapeutic agent, the nature of the medical device, and so forth.

Once formed, the finished medical device can be sterilized chemically (e.g., using ethylene oxide) or by exposure to radiation. The radiation that is used to sterilize the medical devices of the present invention is typically ionizing radiation, such as gamma radiation or electron beam radiation.

It is beneficial in some embodiments to package the medical device in either a vacuum or in an inert atmosphere, for example, in an atmosphere of nitrogen and/or noble gases (e.g. helium, neon, argon, krypton etc.), to prevent oxygen from detrimentally interacting with the device. Beneficial packing materials include barrier materials through which radiation sterilization can be conducted and which have sufficient barrier properties to maintain a vacuum or an inert gas atmosphere. Such barrier materials are well known in the art.

### EXAMPLE

The solvent system selected for use in a given procedure will depend upon the nature of the polymer and phenolic compound selected. In the case of polystyrene-polyisobutylene-polystyrene triblock copolymer (SIBS) in combination with BHT, a preferred solution is one containing (a) 99% tetrahydrofuran and (b) 1% copolymer and paclitaxel (combined).

Solutions are provided that contain: (a) 99 wt % tetrahydrofuran (THF), 0.05 wt % BHT and 0.95 wt % copolymer; (b) 99 wt % tetrahydrofuran (THF), 0.10 wt % BHT and 0.90 wt % polymer; or (c) 99 wt % tetrahydrofuran (THF) and 1.0 wt % copolymer (but no BHT). All solutions are prepared by combining the above ingredients together and mixing thoroughly. The BHT was obtained from Sigma (Sigma B1378). The SIBS triblock copolymer is prepared, for example, as described in United States Patent Application No. 2002/0107330 and U.S. Pat. No. 6,545,097 entitled “Drug delivery compositions and medical devices containing block copolymer,” the disclosure of each of which is hereby incorporated by reference in its entirety.

Each solution is then placed in a syringe pump and fed to a spray nozzle. A stainless steel, balloon expandable stent is mounted onto a holding device parallel to the nozzle and rotated to ensure uniform coverage. Depending on the spray equipment used, either the stent or spray nozzle can be moved while spraying such that the nozzle moves along the stent while spraying for one or more passes. After a coating is formed, the stent is dried, for example, by placing it in a preheated oven.

Subsequent to coating, the stents are sterilized with either electron beam radiation (dose=25 Kgray) or by exposure to ethylene oxide (EtO) using procedures known in the art.

The following are placed in the coronary arteries of juvenile domestic swine: (a) oversized bare metal stents, (b) EtO-sterilized, copolymer-coated, BHT-free stents, (c) electron-beam-sterilized, copolymer-coated, BHT-free stents, (d) electron-beam-sterilized, stents with coating containing 5% BHT and 95% copolymer, and (e) electron beam-sterilized, stents with coating containing 10% BHT and 90% copolymer.

After 28 days, the stents are harvested from the animals and an examination of morphometric % restenosis and neointimal thickening area was conducted. Morphometric analysis is performed on one specimen from each stented segment in a blinded manner. Morphometry is completed using a PC based digital planimetry system. Morphometric % restenosis is determined by neointimal area÷internal elastic lamina (IEL) area×100; while neointimal area is determined by internal elastic lamina (IEL)−injured luminal area. The results are presented in the table to follow:

| | MORPHOMETRIC % | NEOINTIMAL |
|---|---|---|
| GROUPS | RESTENOSIS | AREA (MM²) |
| Bare Stents (n = 12) | 26.5 ± 8.1 | 2.5 ± 0.9 |
| Polymer + EtO (n = 6) | 42.0 ± 13.2 | 4.2 ± 1.3 (p = 0.01) |
| | (p = 0.002) | |
| Polymer + Ebeam (n = 8) | 36.7 ± 10.5 (p = 0.02) | 3.7 ± 1.2 (p = 0.0) |
| Polymer + 5% BHT + | 26.6 ± 3.7 (p = 0.98) | 2.6 ± 0.3 (p = 0.89) |
| Ebeam (n = 6) | | |
| Polymer + 10% BHT + | 24.4 ± 3.0 (p = 0.67) | 2.5 ± 0.3 (p = 0.94) |
| Ebeam (n = 6) | | |

Statistical methods are as follows: Data are presented as mean±S.D. Comparisons between the control (bare stent) and treated stents were made using unpaired t-test. Improvement in morphometic % restenosis and neointimal area, relative to bare stents, was not considered to be statistically significant for values of p≦0.05.

As seen from the above, after 28 days, histology demonstrated a significant increase in restenosis and neointimal thickening for stents coated with copolymer alone, as compared with bare metal stents. Copolymer stents containing 5% BHT and 10% BHT, on the other hand, compare favorably with the bare metal stents.

Hence, it has been shown that the presence of BHT enhances stent biocompatibility, suggesting the possible role of anti-oxidation in reducing undesirable polymer-induced effects on the media and neointima in the porcine coronary artery model.

Although various embodiments are specifically illustrated and described herein, it will be appreciated that modifications and variations of the present invention are covered by the above teachings and are within the purview of the appended claims without departing from the spirit and intended scope of the invention.

1. A vascular medical device comprising a polymeric region and a phenolic compound, said polymeric region regulating the release of said phenolic compound from said device, and said polymeric region comprising a polymer that comprises a monomer selected from a vinyl aromatic monomer, an alkene monomer, or both a vinyl aromatic monomer and an alkene monomer.

2. The vascular medical device of claim 1, wherein said medical device is a stent.

3. The vascular medical device of claim 2, wherein said polymeric region is in the form of a polymeric layer disposed over metallic stent substrate.

4. The vascular medical device of claim 1, wherein said medical device is a catheter.

5. The vascular medical device of claim 4, wherein said catheter is a balloon catheter.

6. The vascular medical device of claim 1, wherein said phenolic compound is released from said medical device in an amount that is effective to reduce the amount of neointimal thickening that otherwise arises upon insertion or implantation of said medical device into the vasculature in the absence of said phenolic compound

7. The vascular medical device of claim 1, wherein said phenolic compound is a hindered phenol.

8. The vascular medical device of claim 1, wherein said phenolic compound is butylated hydroxytoluene.

9. The vascular medical device of claim 1, wherein said polymeric region is in the form of a polymeric layer disposed over an underlying medical device substrate.

10. The vascular medical device of claim 9, wherein said phenolic compound is disposed within said polymeric layer.

11. The vascular medical device of claim 9, wherein said phenolic compound is disposed beneath said polymeric layer.

12. The vascular medical device of claim 1, wherein said polymer comprises an alkene monomer.

13. The vascular medical device of claim 1, wherein said polymer is a copolymer that comprises an isobutylene monomer.

14. The vascular medical device of claim 1, wherein said polymer comprises a vinyl aromatic monomer.

15. The vascular medical device of claim 1, wherein said polymer is a copolymer that comprises a styrene monomer.

16. The vascular medical device of claim 1, wherein said polymer is a copolymer that comprises an alkene monomer and a vinyl aromatic monomer.

17. The vascular medical device of claim 1, wherein said polymer is a copolymer that comprises an isobutylene monomer and a styrene monomer.

18. The vascular medical device of claim 1, wherein said polymer is a block copolymer that comprises a polystyrene block and a polyisobutylene block.

19. The vascular medical device of claim 1, wherein said polymer is a polystyrene-polyisobutylene-polystyrene triblock copolymer.

20. A vascular medical device comprising a polymeric region and a phenolic compound, said polymeric region regulating the release of said phenolic compound from said device, and said polymeric region comprising a biostable polymer displaying at least one glass transition temperature below 25° C.

21. The vascular medical device of claim 20, wherein said biostable polymer is a biostable copolymer.

22. The medical device of claim 21, wherein said biostable copolymer is a block copolymer comprising a poly(alkene) block.

23. The vascular medical device of claim 21, wherein said biostable copolymer further displays at least one T_{g} above 50° C.

24. The medical device of claim 23, wherein said biostable copolymer is a block copolymer comprising a poly(alkene) block and a poly(vinyl aromatic) block.
